(19) United States
(12) Patent Application Publication
(10) Pub. No.: US 2026/0115385 A1
(43) Pub. Date: Apr. 30, 2026

(54) BUTTON ACTUATED AUTOINJECTOR

(71) Applicant: HALOZYME, INC., SAN DIEGO, CA (US)

(72) Inventors: Ryan WANGSNES, S MINNEAPOLIS, MN (US); Kevin SWANSON, PLYMOUTH, MN (US); Mike TRAVANTY, WAYZATA, MN (US); Todd LEADENS, MAPLE GROVE, MN (US)

(21) Appl. No.: 18/846,136

(22) PCT Filed: Dec. 21, 2023
(86) PCT No.: PCT/US2023/085307

§ 371 Date: Sep. 11, 2024

(51) Int. Cl. A61M 5/20; A61M 5/315; A61M 5/32

(52) U.S. Cl. CPC A61M 5/2033; A61M 5/31501; A61M 5/31571; A61M 5/3158; A61M 5/3202; A61M 2005/2073

(57) ABSTRACT

An injector includes a housing that has a proximal end and a distal end that extends along a longitudinal axis. A medicament container contains a volume of a medicament. A button coupled to the housing is rotatable about the longitudinal axis between a locked position and an unlocked position and movable along the longitudinal axis between a pre-firing position and a firing position. A latch is at least partially disposed within the button, a ram is movable relative to the latch, and an energy source urges the ram distally along the longitudinal axis. The ram is prevented from moving relative to the latch by the button in the pre-firing position. Rotation of the button to the unlocked position and movement of the button distally along the longitudinal axis relative to the housing into the firing position allows the ram to move relative to the latch.

### TECHNICAL FIELD

The present disclosure generally relates to button actuated autoinjectors, and in some embodiments, button actuated triggering devices for the delivery of large volumes of medicament for sub skin surface penetration.

### SUMMARY

In certain embodiments, an injector comprises: a housing having a proximal end and a distal end extending along a longitudinal axis; a medicament container containing a volume of a medicament; a button coupled to the housing, the button rotatable about the longitudinal axis between a locked position and an unlocked position and movable along the longitudinal axis between a pre-firing position and a firing position; a latch at least partially disposed within the button; a ram movable relative to the latch; and an energy source configured to urge the ram distally along the longitudinal axis. In certain embodiments, the ram is prevented from moving relative to the latch by the button in the pre-firing position. In certain embodiments, rotation of the button to the unlocked position and movement of the button distally along the longitudinal axis relative to the housing into the firing position allows the ram to move relative to the latch.

In certain embodiments, the button includes a button body, a button aperture extending through the button body, and a fin extending distally from a proximal end of the button body. In certain embodiments, the latch includes a latch body, a crown defined on a proximal end of the latch body configured to engage the fin of the button, and an engagement feature extending from the latch body, the engagement feature movable between an engaged position and a disengaged position. In certain embodiments, at least a portion of the fin engages the crown when the button is in the locked position and in the unlocked position.

In certain embodiments, the ram includes a ram body, and a ram aperture extending through the ram body, the ram aperture configured to receive at least a portion of the engagement feature when the button is in the pre-firing position. In certain embodiments, the engagement feature comprises: a flexible arm extending along the longitudinal axis; and a protrusion extending radially therefrom. In certain embodiments, the protrusion extends radially inward from an end of the flexible arm. In certain embodiments, rotation of the button into the unlocked position aligns the button aperture with the engagement feature. In certain embodiments, distal movement of the button into the firing position allows the engagement feature to flex radially outward and extend at least partially through the button aperture.

In certain embodiments, the medicament container includes a luer lock extending from a distal end thereof, the injector further including a plunger disposed within the luer lock. In certain embodiments, the ram engages the plunger thereby moving the plunger relative to the medicament container when the button is moved into the firing position. In certain embodiments, movement of the plunger relative to the medicament container ejects the volume of the medicament from the medicament container through the luer lock.

In certain embodiments, the injector further comprises: a needle coupled to the luer lock, the needle in fluid communication with the medicament container. In certain embodiments, the injector further comprises: a luer cap removably coupled to the luer lock in a storage configuration, wherein the luer cap is fully recessed within the housing. In certain embodiments, the button is prevented from moving proximally relative to the housing after the button is moved from the pre-firing position to the firing position. In certain embodiments, the volume of the medicament is at least 10 mL. In certain embodiments, the latch is fixed relative to the housing, and the medicament container is fixed relative to the housing.

In certain embodiments, a method of delivering a medicament to a subject using an injector, the injector comprising a housing having a proximal end and a distal end extending along a longitudinal axis, a medicament container containing a volume of a medicament, a button coupled to the housing, the button rotatable about the longitudinal axis between a locked position and an unlocked position and movable along the longitudinal axis between a pre-firing position and a firing position, a latch at least partially disposed within the button, a ram movable relative to the latch, and an energy source configured to urge the ram distally along the longitudinal axis, the method comprises: decoupling a luer cap from a luer lock of the medicament container with a luer cap remover, the luer cap fully recessed within the housing; coupling a needle to the luer lock, the needle in fluid communication with the medicament container; rotating the button to the unlocked position; inserting at least a portion of the needle into the subject; moving the button distally relative to the housing into the firing position; ejecting the volume of the medicament through the needle into the subject; and removing the needle from the subject.

In certain embodiments, the ram is prevented from moving relative to the latch by the button in the pre-firing position. In certain embodiments, rotation of the button to the unlocked position and movement of the button distally along the longitudinal axis relative to the housing into a firing position allows the ram to move relative to the latch.

In certain embodiments, the method may further comprise: rotating the button into the unlocked position to align a button aperture of the button with an engagement feature of the latch. In certain embodiments, the method may further comprise: moving the button distally into the firing position thereby allowing the engagement feature to flex radially outward and extend at least partially through the button aperture.

In certain embodiments, the method may further comprise: the ram engages a plunger disposed in the medicament device thereby moving the plunger relative to the medicament container when the button is moved into the firing position. In certain embodiments, the method may further comprise: moving the plunger relative to the medicament container ejects the volume of the medicament from the medicament container through the needle. In certain embodiments, the method may further comprise: the button is prevented from moving proximally relative to the housing after the button is moved to the firing position.

### BRIEF DESCRIPTION OF THE DRAWINGS

The foregoing summary, as well as the following detailed description of embodiments of the button actuated autoinjector, will be better understood when read in conjunction with the appended drawings of an exemplary embodiment. It should be understood, however, that the invention is not limited to the precise arrangements and instrumentalities shown.

In the drawings:

FIG. 1 is a front view of a button actuated autoinjector in accordance with an exemplary embodiment of the present invention;

FIG. 2 is a side view of the autoinjector of FIG. 1;

FIG. 3 is a front cross-sectional view of the autoinjector of FIG. 1;

FIG. 4 is a side cross-sectional view of the autoinjector of FIG. 1;

FIG. 5 is a front partial and semi-transparent view of the autoinjector of FIG. 1;

FIG. 6 is a front partial and semi-transparent view of the autoinjector of FIG. 1;

FIG. 7 is a front cross-sectional partial view of the autoinjector of FIG. 1 with the button in the pre-firing position;

FIG. 8 is a side cross-sectional partial view of the autoinjector of FIG. 1 with the button in the pre-firing position;

FIG. 9 is a front cross-sectional partial view of the autoinjector of FIG. 1 with the button in the firing position;

FIG. 10 is a side cross-sectional partial view of the autoinjector of FIG. 1 with the button in the firing position;

FIG. 11 is a front view of a button of the autoinjector of FIG. 1;

FIG. 12 is a bottom perspective cross-sectional view of the button of the autoinjector of FIG. 1;

FIG. 13 is a top perspective view of a latch of the autoinjector of FIG. 1;

FIG. 14 is a side cross-sectional view of the latch of the autoinjector of FIG. 1;

FIG. 15 is a bottom perspective view of a ram of the autoinjector of FIG. 1;

FIG. 16 is a side view of the ram of the autoinjector of FIG. 1;

FIG. 17 is a front view of a medicament container of the autoinjector of FIG. 1 with a needle coupled thereto;

FIG. 18 is a front cross-sectional view of the medicament container of the autoinjector of FIG. 1 with a plunger disposed therein and a needle coupled thereto;

FIG. 19 is a front view of a front housing of the autoinjector of FIG. 1;

FIG. 20 is a side view of the front housing of the autoinjector of FIG. 1;

FIG. 21 is a front view of a luer cap remover of the autoinjector of FIG. 1;

FIG. 22 is a front view of a needle shield of the autoinjector of FIG. 1;

FIG. 23A is a front view of a luer cap remover disposed in a distal end of the autoinjector of FIG. 1;

FIG. 23B is a front view of a luer cap being removed from the autoinjector of FIG. 1;

FIG. 24A is a front view of a needle and needle shield being introduced to the autoinjector of FIG. 1;

FIG. 24B is a front view of the needle being attached to the autoinjector of FIG. 1; and

FIG. 24C is a front view of the needle shield being removed from the needle and the autoinjector of FIG. 1.

### DETAILED DESCRIPTION

The present disclosure generally relates to autoinjectors, button actuated triggering devices, that are used in the delivery of large volumes of injectable fluid, such as medicament, (e.g., 10 ml or larger doses) for sub skin surface penetration (e.g., subcutaneous and intramuscular injections). Devices that deliver large volumes of medicament are often prone to configuration restrictions as the delivery volume of the medicament increases, such as spring force limitations and syringe container breakage. Due to the current configurations of overall device design, and the limitations of the biological uptake factors that limit injection speed and volume, injection devices are often held for long durations, and often result in other delivery methods being used such as on-body delivery systems which are attached to the patient during delivery.

Increasingly, more biologics are allowing for at-home administration for patients. However, for many biologics, large doses and the resulting large volume of medicament that must be delivered often precludes self-administration because of the length of time required to hold the delivery device in place. Protein hyper-concentration may be used to reduce injection volume in certain instances, but the resulting medicament often has a much higher viscosity than the traditional biologics. High powered injectors may allow delivery of these hyper-concentrated proteins. However, typical handheld injector designs only allow for up to 2.5 mL of a viscous medicament to be injected within 30 seconds.

With the development of enzymes which locally degrade hyaluronan (HA) in the subcutaneous (SC) space, thereby temporarily removing a barrier to fluid flow, the traditional limitations of biological uptake factors that limit injection speed and volume may be reduced. As such, higher volumes of viscous medicaments may be delivered. Accordingly, there is a need to provide a handheld device capable of delivering a large dose (e.g., 3 mL, 5 mL, 10 mL, 20 mL and up to 50 mL) of a viscous medicament in a delivery time appropriate for a handheld device.

Drug delivery technology of large doses of a viscous medicament is currently based on the proprietary recombinant human hyaluronidase PH20 enzyme (e.g., rHuPH20; Halozyme, Inc.) that facilitates SC delivery of co-administered therapeutics. rHuPH20 works by degrading the HA, which plays a role in resistance to bulk fluid flow in the SC space, limiting large volume SC drug delivery, dispersion, and absorption. Co-administration of rHuPH20 with injectable therapies can overcome administration time and volume barriers associated with existing SC therapeutic formulations, and has been shown to reduce the burden on patients and healthcare providers compared with intravenous formulations. rHuPH20 has countless applications in the current field of injectable therapies by increasing the dispersion and absorption of other injected drugs, such as anticancer therapies (e.g., trastuzumab and rituximab), immunodeficiency treatment, in subcutaneous urography for improving resorption of radiopaque agents, and fluid delivery for rehydration.

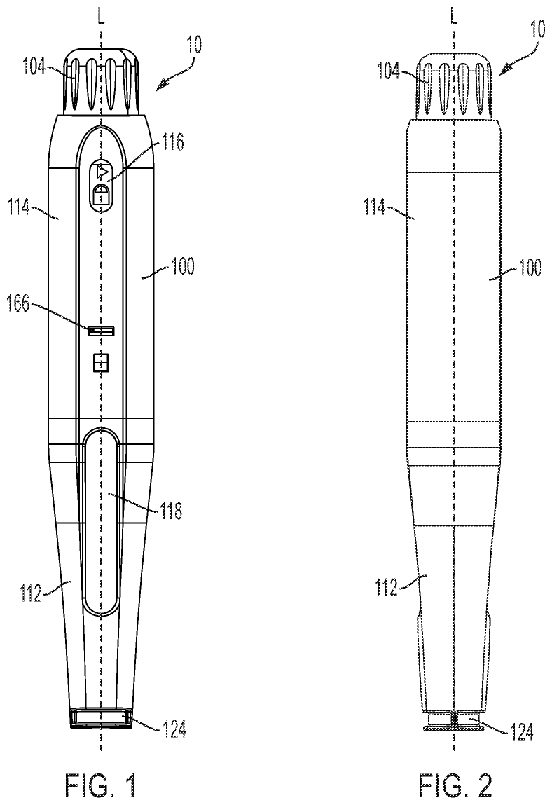

Referring to the drawings in detail, wherein like reference numerals indicate like elements throughout, there is shown in FIGS. 1-24C an autoinjector, generally designated 10, in accordance with an exemplary embodiment of the present invention.

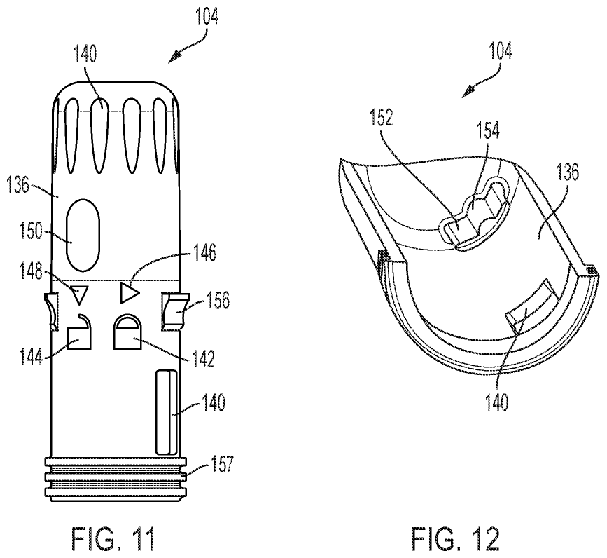

As shown in FIGS. 1-4, the injector 10 may include a housing 100, a medicament container 102, a button 104, a latch 106, a ram 108 and an energy source 110. The housing 100 may have a proximal end and a distal end extending along a longitudinal axis L. The housing 100 may be configured for allowing a user to grip or handle the injector 10. The housing 100 may be shaped to fit into a user's hand for single-handed function. The housing 100 may have a generally oval cross-section to help position the injector 10 in the user's hand. The housing 100 may further include a ridge extending along the longitudinal axis L of the housing (as shown in FIG. 1) to help align or position the injector 10 in the user's hand. The housing 100 may substantially house the components shown in FIGS. 11-18.

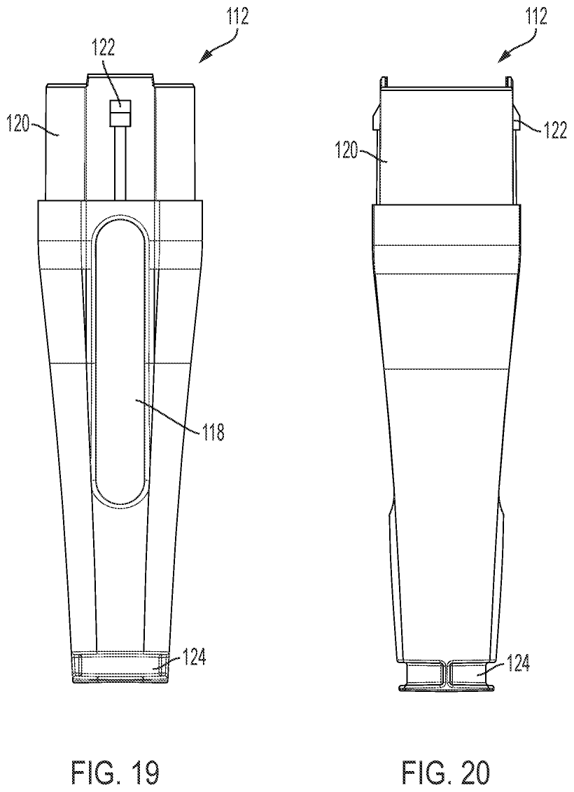

The housing 100 may comprise a front housing 112 and a rear housing 114. The front housing 112 and rear housing 114 may be coupled together by snap-fit coupling. In some embodiments, the front housing 112 and rear housing 114 are coupled using an adhesive or other suitable means to prevent separation during use of the injector 10. The rear housing 114 may have a generally oval cross section with open proximal and distal ends. The proximal end of the rear housing 114 may have a generally circular opening sized and shaped to allow at least a portion of the button 104 to pass therethrough. The distal end of the rear housing 114 may have an opening sized and shaped to receive at least a portion of the front housing 112 therein. As shown in FIG. 19, the front housing 112 may have a neck 120 extending from a proximal end thereof. The neck 120 may include a neck protrusion 122 extending radially therefrom. The neck protrusion 122 may be received by a rear housing recess (not shown) when the neck 120 is disposed in the rear housing 114.

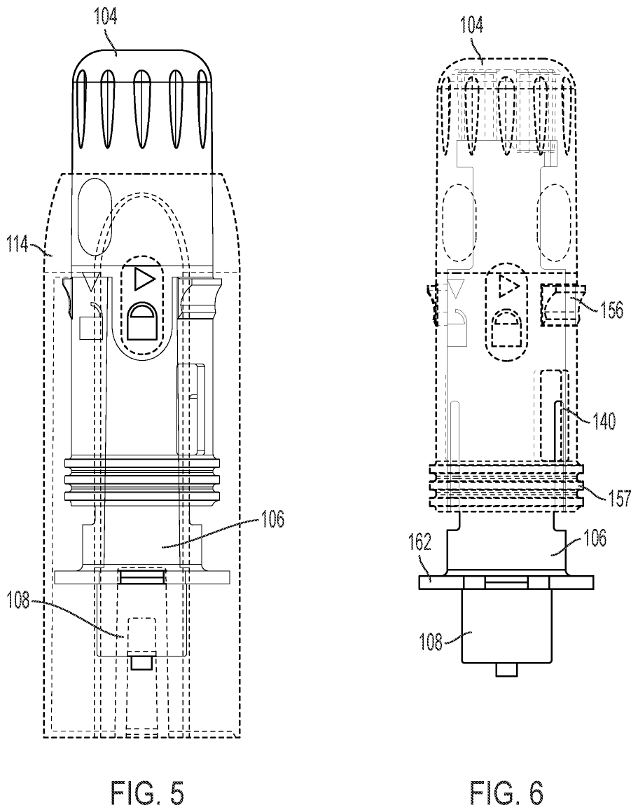

As shown in FIG. 5, the rear housing 114 may include a status window 116 extending therethrough. The status window 116 may be a generally oval shape and sized to display an indicia of the button 104, as described in more detail below. The status window 116 may be located at a proximal end of the rear housing. The status window 116 is shown in a front side of the rear housing 114 in FIG. 1, but it should be appreciated that the status window 116 can be located on any side of the rear housing 114.

As shown in FIGS. 1 and 19, the front housing 112 may be tapered and include a medicament window 118 extending therethrough. The medicament window 118 may include a transparent film or may be empty. The medicament window 118 allows a user to visually inspect the contents of the medicament container 102 before, during and after use of the injector 10. In some embodiments, the medicament window 118 may include one or more lines or symbols to identify the volume of medicament in the medicament container 102. The front housing 112 may include a rimmed distal end 124, as shown in FIGS. 19-20. The rimmed distal end 124 may be molded at varying lengths to accommodate needles of different sizes to ensure proper injection depth.

In some embodiments, the rimmed distal end 124 may be shaped and sized to allow approximately 7 mm of the needle 132 to extend therethrough. In some embodiments, the rimmed distal end 124 may be shaped and sized to allow approximately 7.5 mm of the needle 132 to extend therethrough. In some embodiments, the rimmed distal end 124 may be shaped and sized to allow approximately 8 mm of the needle 132 to extend therethrough. In some embodiments, the rimmed distal end 124 may be shaped and sized to allow approximately 8.5 mm of the needle 132 to extend therethrough. In some embodiments, the rimmed distal end 124 may be shaped and sized to allow approximately 9 mm of the needle 132 to extend therethrough. In some embodiments, the rimmed distal end 124 may be shaped and sized to allow approximately 9.5 mm of the needle 132 to extend therethrough. In some embodiments, the rimmed distal end 124 may be shaped and sized to allow approximately 10 mm of the needle 132 to extend therethrough. In some embodiments, the rimmed distal end 124 may be shaped and sized to allow approximately 10.5 mm of the needle 132 to extend therethrough. In some embodiments, the rimmed distal end 124 may be shaped and sized to allow approximately 11 mm of the needle 132 to extend therethrough. In some embodiments, the rimmed distal end 124 may be shaped and sized to allow approximately 11.5 mm of the needle 132 to extend therethrough. In some embodiments, the rimmed distal end 124 may be shaped and sized to allow approximately 12 mm of the needle 132 to extend therethrough. In some embodiments, the rimmed distal end 124 may be shaped and sized to allow approximately 12.5 mm of the needle 132 to extend therethrough. In some embodiments, the rimmed distal end 124 may be shaped and sized to allow approximately 13 mm of the needle 132 to extend therethrough.

The medicament container 102 containing a medicament may be disposed within the housing 100. The medicament container 102 may be fixed relative to the housing 100. As used herein, the medicament may comprise drugs, biologics, solutions, gels, suspensions or other substances that may be delivered via a syringe or needle, and such terms may be used interchangeably as appearing in the specification and claims. The medicament container 102 may be a prefilled syringe. In some embodiments, the medicament container 102 may be an insertable medicament cartridge. In some embodiments, the medicament container 102 may be filled using a vial-and-needle or other appropriate filling method. In one embodiment, the medicament container 102 is one of a prefilled cartridge, prefilled staked needle syringe, vial, or other injectable fluid containing vessel. The medicament container 102 has a distal portion and a proximal portion opposite the distal portion.

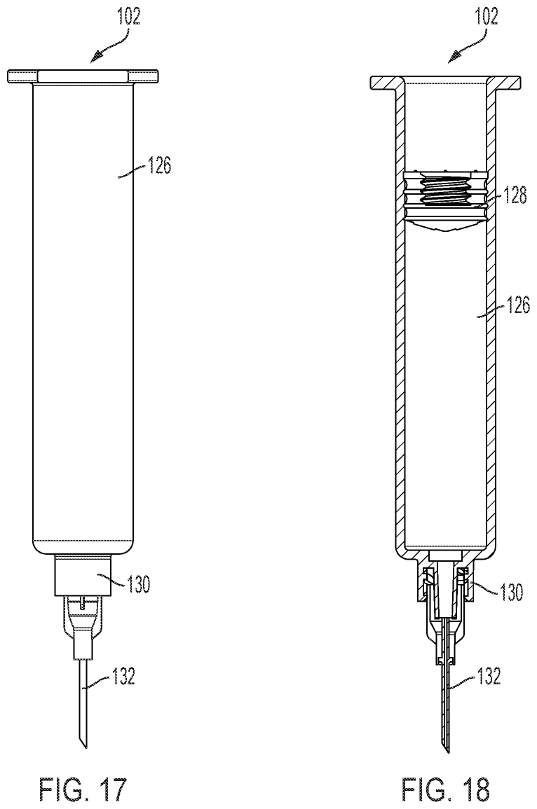

As shown in FIGS. 17-18, the medicament container 102 may comprise a container portion 126 defining a fluid chamber containing the medicament. In one embodiment, the container portion 126 of the medicament container 102 has a maximum volume of approximately 10 mL. In one embodiment, the container portion 126 of the medicament container 102 has a maximum volume selected from approximately: 3 mL, 3.5 mL, 4 mL, 4.5 mL, 5.5 mL, 6 mL, 6.5 mL, 7 mL, 7.5 mL, 8 mL, 8.5 mL, 9 mL, 9.5 mL, 10 mL, 10.5 mL, 11 mL, 11.5 mL, 12 mL, 12.5 mL, 13 mL, 13.5 mL, 14 mL, 14.5 mL, 15 mL, 15.5 mL, 16 mL, 16.5 mL, 17 mL, 17.5 mL, 18 mL, 18.5 mL, 19 mL, 19.5 mL, 20 mL. 25 mL, 30 mL, 35 mL, 40 mL, 45 mL, and 50 mL.

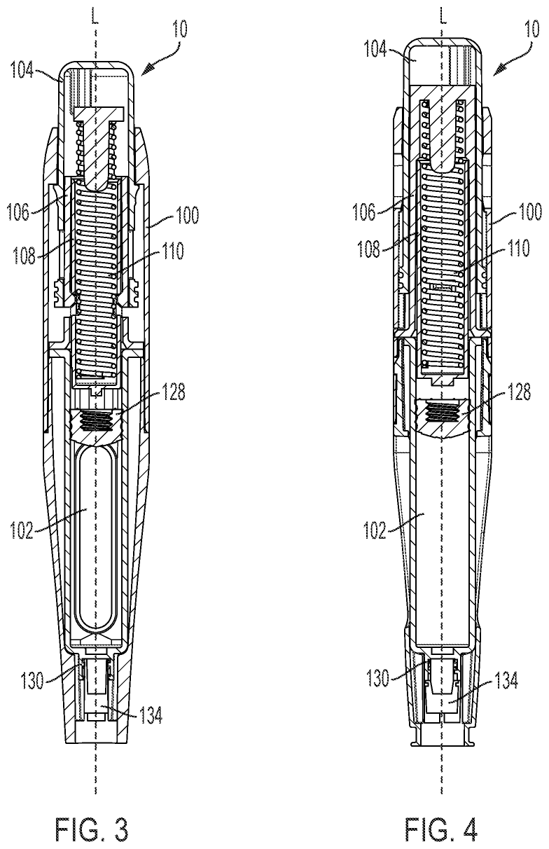

As shown in FIGS. 3, 4 and 18, the medicament container 102 may further comprise a plunger 128 movable within the container portion 126 relative to the medicament container 102. Prior to use or triggering of the injector 10, the plunger 128 may be disposed at a distal end of the container portion 126. The plunger 128 may seal the medicament in the container portion 126. The plunger 128 may be made of a rubber material. In one embodiment, the plunger 128 is made of a plastic, butyl rubber, polyisoprene, polytetrafluorethylene, high density polyethylene or other thermoset elastomers.

A flange 130 may extent outwardly from a distal portion of the medicament container 102. In one embodiment, the flange 130 is a luer. In one embodiment, the flange 130 is a luer lock. As shown in FIGS. 3-4, the flange 130 may include a luer cap 134 removably coupled thereto. The luer cap 134 may be coupled to the flange 130 in a storage configuration. The luer cap 134 may be fully disposed within the housing 100. In one embodiment, the luer cap 134 may be at least partially disposed within the housing 100.

In one embodiment, as shown in FIGS. 17-18, the flange 130 may be couplable to a needle 132 in fluid communication with the medicament in the container portion 126. The needle 132 may include a connector 133 at a proximal end thereof. The connector 133 may be a threaded member shaped and sized to mate with the flange 130. In some embodiments, the connector 133 includes deformable ribs that engage the flange 130 to couple the needle 132 thereto. The needle 132 may be a 25 gauge needle. In some embodiments, the needle 132 may be a 15 gauge needle, a 16 gauge needle, a 17 gauge needle, a 18 gauge needle, a 19 gauge needle, a 20 gauge needle, a 21 gauge needle, a 22 gauge needle, a 23 gauge needle, a 24 gauge needle, a 25 gauge needle, a 26 gauge needle, a 27 gauge needle, a 28 gauge needle, a 29 gauge needle, a 30 gauge needle, a 31 gauge needle, a 32 gauge needle, a 33 gauge needle, a 34 gauge needle, or a 35 gauge needle.

In one embodiment, the flange 130 may be couplable to a tubing set (not shown) in fluid communication with the medicament in the container portion 126. The needle 132 may be coupled to the flange 130 by the user, as described in more detail below. In one embodiment, a staked needle 27 may be pre-attached and extending from the distal portion of the medicament container 102.

The injector 10 may be configured to deliver the entire amount or a portion of the medicament within medicament container 102. The medicament contained in the medicament container 102 may have a volume of approximately 10 mL. In one embodiment, the medicament contained in the medicament container 102 corresponds to a volume selected from:
a) 3 mL to 5 mL, 3 mL to 10 mL, 3 mL to 15 mL, 3 mL to 20 mL, 3 mL to 25 mL, 3 ml to 30 mL, 3 mL to 35 mL, 3 mL to 40 mL, 3 mL to 45 mL, 3 mL to 50 mL, 5 mL to 10 mL, 5 mL to 15 mL, 5 mL to 20 mL, 5 mL to 25 mL, 5 mL to 30 mL, 5 mL to 35 mL, 5 mL to 40 mL; 5 mL to 45 mL, 5 mL to 50 mL, 10 mL to 15 mL; 10 mL to 20 mL; 10 mL to 25 mL; 10 mL to 30 mL; 10 mL to 35 mL; 10 mL to 40 mL, 10 mL to 50 mL;
b) about 3 mL to about 5 mL, about 3 mL to about 10 mL, about 3 mL to about 15 mL, about 3 mL to about 20 mL, about 3 mL to about 25 mL, about 3 ml to about 30 mL, about 3 mL to about 35 mL, about 3 mL to about 40 mL, about 3 mL to about 45 mL, about 3 mL to about 50 mL, about 5 mL to about 10 mL, about 5 mL to about 15 mL, about 5 mL to about 20 mL, about 5 mL to about 25 mL, about 5 mL to about 30 mL, about 5 mL to about 35 mL, about 5 mL to about 40 mL; about 5 mL to about 45 mL, about 5 mL to about 50 mL, about 10 mL to about 15 mL; about 10 mL to about 20 mL; about 10 mL to about 25 mL; about 10 mL to about 30 mL; about 10 mL to about 35 mL; about 10 mL to about 40 mL, about 10 mL to about 50 mL;
c) at least about 3 mL, at least about 3.5 mL, at least about 4 mL, at least about 4.5 mL, at least about 5.5 mL, at least about 6 mL, at least about 6.5 mL, at least about 7 mL, at least about 7.5 mL, at least about 8 mL, at least about 8.5 mL, at least about 9 mL, at least about 9.5 mL, at least about 10 mL, at least about 10.5 mL, at least about 11 mL, at least about 11.5 mL, at least about 12 mL, at least about 12.5 mL, at least about 13 mL, at least about 13.5 mL, at least about 14 mL, at least about 14.5 mL, at least about 15 mL, at least about 15.5 mL, at least about 16 mL, at least about 16.5 mL, at least about 17 mL, at least about 17.5 mL, at least about 18 mL, at least about 18.5 mL, at least about 19 mL, at least about 19.5 mL, at least about 20 mL, at least about 25 mL, at least about 30 mL, at least about 35 mL, at least about 40 mL, at least about 45 mL, at least about 50 mL; and
d) at least 3 mL, at least 3.5 mL, at least 4 mL, at least 4.5 mL, at least 5.5 mL, at least 6 mL, at least 6.5 mL, at least 7 mL, at least 7.5 mL, at least 8 mL, at least 8.5 mL, at least 9 mL, at least 9.5 mL, at least 10 mL, at least 10.5 mL, at least 11 mL, at least 11.5 mL, at least 12 mL, at least 12.5 mL, at least 13 mL, at least 13.5 mL, at least 14 mL, at least 14.5 mL, at least 15 mL, at least 15.5 mL, at least 16 mL, at least 16.5 mL, at least 17 mL, at least 17.5 mL, at least 18 mL, at least 18.5 mL, at least 19 mL, at least 19.5 mL, at least 20 mL, at least 25 mL, at least 30 mL, at least 35 mL, at least 40 mL, at least 45 mL, at least 50 mL.

Flow rate of the injector 10 is heavily dependent on the viscosity and volume of the medicament. However, the injector 10 may deliver the full volume of the medicament at a rate of approximately 0.33 mL/sec. The injector 10 may deliver 10 mL of the medicament at a rate of 0.33 mL/sec. In one embodiment, the injector 10 delivers the full deliverable volume of the medicament at a rate of:
a) 0.5 mL/10 sec., 0.75 mL/10 sec., 1 mL/10 sec., 1.25 mL/10 sec., 1.5 mL/10 sec., 1.75 mL/10 sec, 2 mL/10 sec., 2.25 mL/10 sec, 2.5 mL/10 sec., 2.75 mL/10 sec, 3 mL/10 sec., 3.25 mL/10 sec, 3.5 mL/10 sec., 3.75 mL/10 sec, 4 mL/10 sec., 4.25 mL/10 sec, 4.5 mL/10 sec., 4.75 mL/10 sec, 5 mL/10 sec;
b) 2 mL/30 sec., 2.5 mL/30 sec., 3 mL/30 sec., 3.5 mL/30 sec., 4 mL/30 sec., 4.5 mL/30 sec., 5 mL/30 sec., 5.5 mL/30 sec., 6 mL/30 sec., 6.5 mL/30 sec., 7 mL/30 sec., 7.5 mL/30 sec., 8 mL/30 sec., 8.5 mL/30 sec., 9 mL/30 sec., 9.5 mL/30 sec., 10 mL/30 sec., 10.5 mL/30 sec.; and
c) 4 mL/min, 5 mL/min, 6 mL/min, 7 mL/min, 8 mL/min, 9 mL/min, 10 mL/min, 11 mL/min, 12 mL/min, 13 mL/min, 14 mL/min, 15 mL/min, 16 mL/min, 17 mL/min, 18 mL/min, 19 mL/min, 20 mL/min, 21 mL/min.

The viscosity of the medicament may be between 1-20 cP. In one embodiment, the medicament may have a viscosity of:
a) 1 cP, 2 cP, 3 cP, 4 cP, 5 cP, 6 cP, 7 cP, 8 cP, 9 cP, 10 cP, 11 cP, 12 cP, 13 cP, 14 cP, 15 cP, 16 cP, 17 cP, 18 cP, 19 cP, 20 cP; and
b) between 1-20 cP, between 2-19 cP, between 3-18 cP, between 4-17 cP, between 5-16 cP, between 6-15 cP, between 7-14 cP, between 8-13 cP, between 9-12 cP, between 10-11 cP.

In some embodiments, the user's experience is improved if the injector 10 delivers the full volume of the medicament as fast as possible. A faster delivery may result in less pain and discomfort for the patient. The injector 10 may deliver the full deliverable volume of the medicament in less than 5 seconds. In some embodiments, the injector 10 may deliver the full deliverable volume of the medicament in less than 10 seconds, less than 15 seconds, less than 20 seconds, less than 25 seconds, less than 30 seconds, less than 35 seconds, less than 40 seconds, less than 45 seconds, less than 50 seconds, less than 55 seconds, less than 60 seconds, less than 70 seconds, less than 80 seconds, less than 90 seconds, less than 100 seconds, less than 110 seconds, or less than 120 seconds. In some embodiments, the injector 10 may deliver the full volume of the medicament in between 5-120 seconds, between 10-110 seconds, between 15-100 seconds, between 20-90 seconds, between 25-80 seconds, between 30-70 seconds, between 35-60 seconds, between 40-55 seconds, or between 45-50 seconds.

As shown in FIGS. 1-10, the button 104 may be coupled to the housing 100. The button 104 may be rotatable about the longitudinal axis L between a locked position and an unlocked position and may be movable along the longitudinal axis L between a pre-firing position and a firing position. The proximal end of the button 104 may be closed and a distal end of the button 104 may be open. As shown in FIGS. 11-12, the button 104 may include a button body 136. The button body 136 may have a generally cylindrical shape.

The button 104 may include a lock indicator 142 thereon for indicating a lock status of the button and/or for indicating the button 104 is in the locked position. The lock indicator 142 may be engraved, etched, printed or molded in the button 104. In one embodiment, the lock indicator 142 is a decal fixed to the button 104 with an adhesive. In one embodiment, the lock indicator 142 is applied onto the button 104 via spray painting, powder coating, silk screen, laser marking, pad printing, or heat staking. The lock indicator 142 may be a graphic of a lock signifying that the button 104 is in the locked position. The lock indicator 142 may be any combination of shapes and/or words.

The button 104 may include an unlock indicator 144 thereon for indicating a lock status of the button and/or for indicating the button 104 is in the unlocked position. The unlock indicator 144 may be engraved, etched, printed or molded in the button 104. In one embodiment, the unlock indicator 144 is a decal fixed to the button 104 with an adhesive. In one embodiment, the unlock indicator 144 is applied onto the button 104 via spray painting, powder coating, silk screen, laser marking, pad printing, or heat staking. The unlock indicator 144 may be a graphic of a lock signifying that the button 104 is in the unlocked position. The unlock indicator 144 may be any combination of shapes and/or words.

The button 104 may further include a rotation indicator 146 for indicating a direction the button 104 is movable about the longitudinal axis L. The rotation indicator 146 may be engraved in the button 104. In one embodiment, the rotation indicator 146 is a decal fixed to the button 104 with an adhesive. In one embodiment, the rotation indicator 146 is applied onto the button 104 via spray painting, powder coating, silk screen, laser marking, pad printing, or heat staking. The rotation indicator 146 may be an arrow signifying the direction the button 104 must be rotated relative to the housing 100 to transition from the locked position to the unlocked position. The rotation indicator 146 may be any combination of shapes and/or words.

The button 104 may further include a translation indicator 148 for indicating a direction the button 104 is movable along the longitudinal axis L. The translation indicator 148 may be engraved in the button 104. In one embodiment, the translation indicator 148 is a decal fixed to the button 104 with an adhesive. In one embodiment, the translation indicator 148 is applied onto the button 104 via spray painting, powder coating, silk screen, laser marking, pad printing, or heat staking. The translation indicator 148 may be an arrow signifying the direction the button 104 must be moved relative to the housing 100 to transition from the pre-firing position to the firing position. The translation indicator 148 may be any combination of shapes and/or words.

The button 104 may further include a lockout indicator 150 for indicating that the injector 10 has been fired and the button 104 is locked in the firing position. The lockout indicator 150 may be engraved in the button 104. In one embodiment, the lockout indicator 150 is a decal fixed to the button 104 with an adhesive. In one embodiment, the lockout indicator 150 is applied onto the button 104 via spray painting, powder coating, silk screen, laser marking, pad printing, or heat staking. The lockout indicator 150 may be an oval shaped and sized to fill at least a portion of the status window 116 of the housing 100. The lockout indicator 150 may be any combination of shapes and/or words.

In one embodiment, the button 104 contains a partial dosing indicator (not shown) of the medicament, such as priming volume in the location of the lock indicator 142. The partial dosing indicator may be engraved, etched, printed or molded in the button 104. In one embodiment, the partial dosing indicator is a decal fixed to the button 104 with an adhesive. In one embodiment, the partial dosing indicator is applied onto the button 24 via spray painting, powder coating, silk screen, laser marking, pad printing, or heat staking.

A proximal end of the button body 136 may include one or more rib 138 extending laterally inward or outward. The one or more rib 138 may increase friction when a user engages the button 104 to rotate the button 104 about the longitudinal axis L. The button 104 may be configured to rotate 45° about the longitudinal axis L. In some embodiments, the button 104 is configured to rotate 20°, 25°, 30°, 35°, 40°, 45°, 50°, 55°, 60°, 65°, 70°, 75°, 80°, 85°, 90°, 100°, 110°, 120°, 130°, 140°, 150, 160°, 170° or 180° about the longitudinal axis L. In some embodiments, the button 104 is configured to rotate between 20°-180°, between 25°-170°, between 30°-160°, between 35°-150°, between 40°-140°, between 45°-130°, between 50°-120°, between 55°-110°, between 60°-100°, between 65°-90°, between 70°-85°, or between 75°-80° about the longitudinal axis L.

As shown in FIGS. 11-12, the button 104 may include a button aperture 140 extending through the button body 136. The button aperture 140 may be located proximate a distal end of the button body 136. The button aperture 140 may be shaped and sized to receive a component of the latch 106, as described in more detail below. The button 104 may include two button apertures 140 spaced apart on the button body 136. In some embodiments, there is one button aperture 140.

As shown in FIG. 12, the button 104 may include a fin 152 extending distally from the proximal end of the button 104. The fin 152 may extend from an interior surface of the button 104. The fin 152 may have a generally curved shape similar to that of the button body 136. The space between the button body 136 and the fin 152 may be the same along a width of the fin 152. The fin may include a ridge 154 extending radially inward along an interior thereof. The ridge 154 may extend along a length of the fin 152. In one embodiment, the ridge 154 may extend from only a portion of the fin 152. The ridge may be a generally rounded shape.

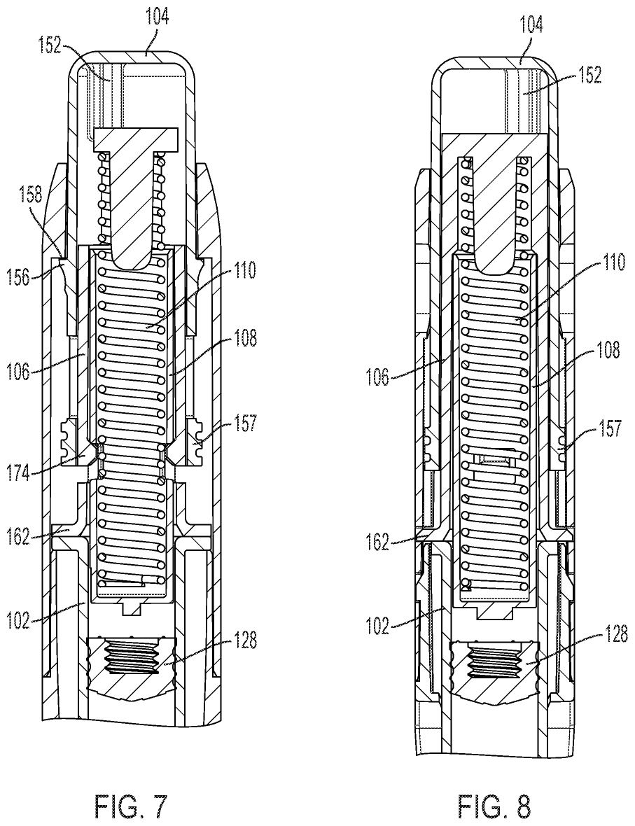

As shown in FIG. 11, the button 104 may include a button protrusion 156 extending radially outward from the button body 136. The button protrusion 156 may be located between the proximal end and the distal end of the button body 136. The button protrusion 156 may be shaped and sized to engage a shelf 158 of the rear housing 114 to prevent the button 104 from moving proximally beyond a predetermined position, as shown in FIG. 7. The button protrusion 156 may have a generally flat proximal end that engages the shelf 158. The interaction of the button protrusion 156 and the shelf 158 may allow the button 104 to smoothly rotate relative to the housing 100. The button protrusion 156 may prevent rotation of the button 104 beyond a predetermined amount when the button protrusion 156 engages an end of the shelf 158. The button 104 may further include a rim 157 extending around the circumference of the button body 136. The rim 157 may prevent deformation of the button 104. In some embodiments, as shown in FIG. 11, there are a plurality of rims 157 extending around the circumference of the button body 136.

As shown in FIG. 6, the latch 106 may be at least partially disposed within the button 104. The button 104 may be allowed to rotate and translate relative to the latch 106. As shown in FIG. 13, the latch 106 may comprise a latch body 160. The latch body 160 may be a generally cylindrical shape. The latch may include a latch collar 162 extending radially outward therefrom. The latch collar 162 may be located at a distal end of the latch body 160. The latch collar 162 may include a collar engagement feature 164 extending radially therefrom. The collar engagement feature 164 may be shaped and sized to be received in a latch receiver 166 of the housing 100. As shown in FIG. 1, the collar engagement feature 164 may be disposed in the latch receiver 166 when the injector 10 is assembled. The latch 106 may be fixed relative to the housing 100.

As shown in FIG. 13, the latch 106 may include a crown 168 defined on a proximal end of the latch body 160. The crown 168 may be configured to engage with the fin 152 of the button. The crown 168 may include one or more detent 170 defined therein. The detent 170 may be shaped and sized to receive the ridge 154 of the fin 152. The crown 168 may further include a surface 172 configured to prevent button 104 from moving distally relative to the latch 106. As shown in FIG. 13, the crown 168 may include a locking detent 170a abutting the surface 172 and an unlocking detent 170b spaced apart from the surface 172. When the ridge 154 is received in the locking detent 170a, the surface 172 may prevent the button 104 from moving along the longitudinal axis L relative to the latch 106. When the ridge 154 is received in the unlocking detent 170b, the button may be able to move along the longitudinal axis L relative to the latch 106.

As shown in FIG. 13, the latch 106 may include an engagement feature 174 extending from the latch body 160. The engagement feature 174 may be moveable between an engaged position and a disengaged position. The engagement feature 174 may include a flexible arm 176 extending along the longitudinal axis L and a protrusion 178 extending radially therefrom. The protrusion 178 may have a generally triangular cross section and may extend radially inward from an end of the flexible arm 176.

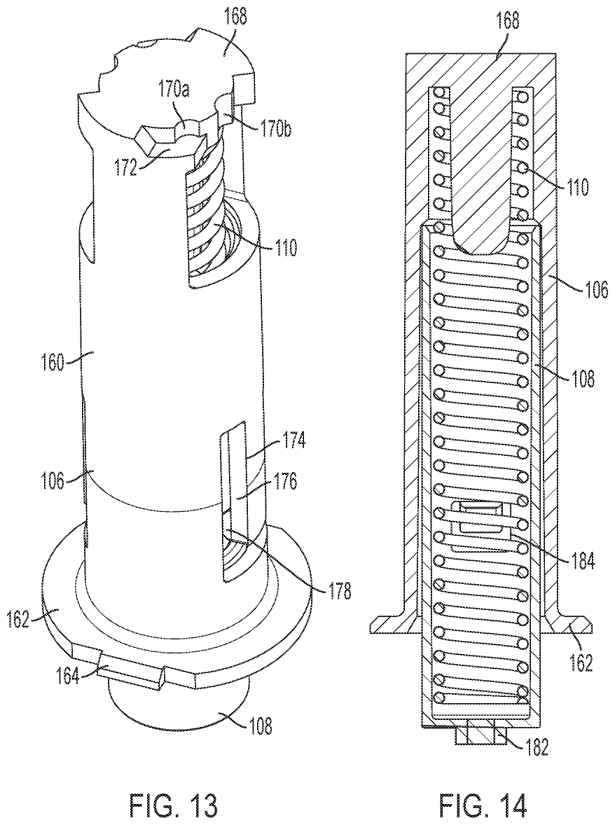

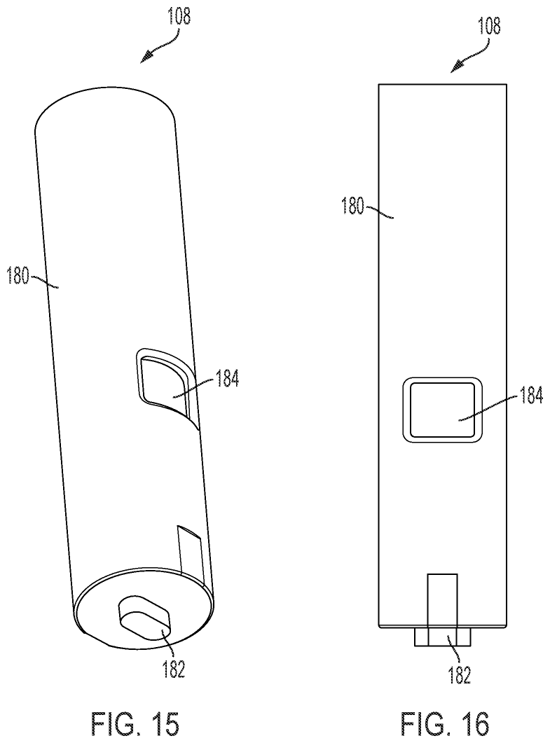

As shown in FIG. 14, the ram 108 may be at least partially disposed in the latch 106. The ram 108 may be fixed relative to the latch 106 when the engagement feature 174 is in the engaged position. The ram 108 may be movable relative to the latch 106 when the engagement feature 174 is in the disengaged position. As shown in FIGS. 15-16, the ram 108 may include a ram body 180. The ram body 180 may be generally cylindrical and may have a closed distal end. The ram body 180 may have a length configured to expel a predetermined volume of the medicament. For example, the length of the ram body 180 may be increased to expel a larger volume of the medicament or may be shortened to expel a smaller volume of the medicament. The distal end of the ram 108 may be configured to interact with the plunger 128 of the medicament container 102. The distal end of the ram 108 may include a ram protrusion 182 extending therefrom. The ram protrusion 182 may be received within the plunger 128. In some embodiments, the distal end of the ram 108 is flush and does not include a ram protrusion 182.

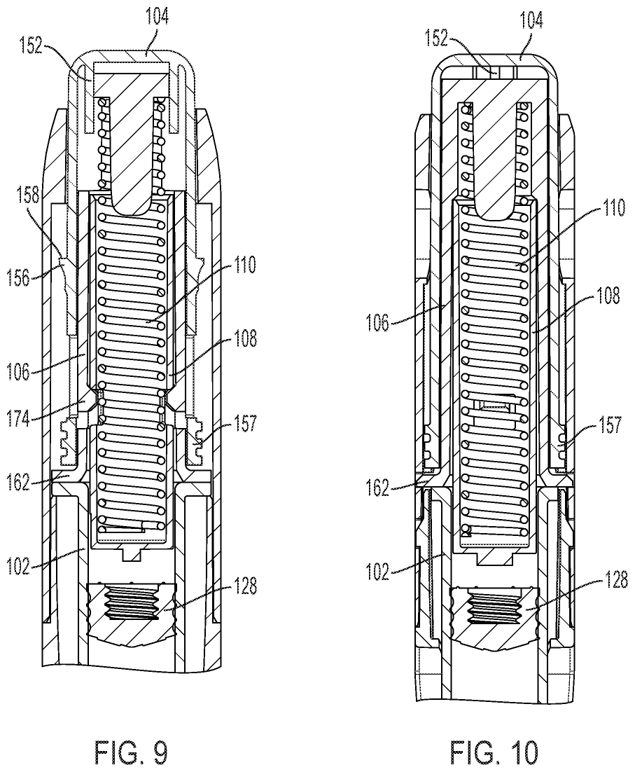

Referring to FIGS. 9-10, the ram 108 may include a ram aperture 184 extending through the ram body 180. The ram aperture 184 may be sized and shaped to receive at least a portion of the engagement feature 174 when the button 104 is in the pre-firing position. The ram aperture 184 may be a generally rectangular shape. As shown in FIG. 13, the ram aperture 184 may receive the protrusion 178 of the engagement feature 174 when the engagement feature is in the engaged position.

The energy source 110 may be configured to urge the ram 108 distally along the longitudinal axis L relative to the latch 106. As shown in FIG. 14, energy source 110 may be disposed at least partially within the ram 108. A proximal end of the energy source 110 may engage a proximal end of the latch 106 and may engage a distal end of the ram 108. In an exemplary embodiment, energy source 110 includes a compression spring, however, other suitable energy source can be used, such as an electric pump, elastomer or compressed-gas spring, or a gas generator, or other suitable energy storage members.

The energy source 110 may be a compression spring. The energy source 110 may have a 5 mm diameter. The energy source 110 may have a 6 mm diameter. The energy source 110 may have a 7 mm diameter. The energy source 110 may have an 8 mm diameter. The energy source 110 may have a 9 mm diameter. The energy source 110 may have a 10 mm diameter. The energy source 110 may have a 11 mm diameter. The energy source 110 may have a 12 mm diameter. The energy source 110 may have a 13 mm diameter. The energy source 110 may have a 14 mm diameter. The energy source 110 may have a 15 mm diameter. The energy source 110 may have a 0.75 mm wire diameter. The energy source 110 may have a 1 mm wire diameter. The energy source 110 may have a 1.25 mm wire diameter. The energy source 110 may have a 1.5 mm wire diameter. The energy source 110 may have a 1.75 mm wire diameter. The energy source 110 may have a 2 mm wire diameter.

The energy source 110 may produce at least 8 lbf of force, at least 9 lbf of force, at least 10 lbf of force, at least 11 lbf of force, at least 12 lbf of force, at least 13 lbf of force, at least 14 lbf of force, at least 15 lbf of force, at least 16 lbf of force, at least 17 lbf of force, at least 18 lbf of force, at least 19 lbf of force, at least 20 lbf of force, at least 21 lbf of force, at least 22 lbf of force, at least 23 lbf of force, at least 24 lbf of force, at least 25 lbf of force, at least 26 lbf of force, at least 27 lbf of force, at least 28 lbf of force, at least 29 lbf of force, or at least 30 lbf of force. The energy source 110 may produce between 8-30 lbf of force, between 9-29 lbf of force, between 10-28 lbf of force, between 11-27 lbf of force, between 12-26 lbf of force, between 13-25 lbf of force, between 14-24 lbf of force, between 15-23 lbf of force, between 16-22 lbf of force, between 17-21 lbf of force, or between 18-20 lbf of force.

The energy source 110 may generate up to 15 lbf of force prior to a triggering event. The energy source 110 may generate up to 17.5 lbf of force prior to a triggering event. The energy source 110 may generate up to 20 lbf of force prior to a triggering event. The energy source 110 may generate up to 22.5 lbf of force prior to a triggering event. The energy source 110 may generate up to 25 lbf of force prior to a triggering event. The energy source 110 may generate up to 27.5 lbf of force prior to a triggering event. The energy source 110 may generate up to 30 lbf of force prior to a triggering event.

The energy source 110 may generate a residual force of 8 lbf of force after expelling the full volume of fluid from the primary container. The energy source 110 may generate a residual force of 10 lbf of force after expelling the full volume of fluid from the primary container. The energy source 110 may generate a residual force of 12 lbf of force after expelling the full volume of fluid from the primary container. The energy source 110 may generate a residual force of 14 lbf of force after expelling the full volume of fluid from the primary container. The energy source 110 may generate a residual force of 16 lbf of force after expelling the full volume of fluid from the primary container. The energy source 110 may generate a residual force of 18 lbf of force after expelling the full volume of fluid from the primary container. The energy source 110 may generate a residual force of 20 lbf of force after expelling the full volume of fluid from the primary container.

The ram 108 may be prevented from moving relative to the latch 106 by the button 104 when the button 104 in the locked position. As shown in FIG. 6, the button aperture 140 and the engagement feature 174 are not aligned along the longitudinal axis L when the button 104 is in the locked position. As such, the button body 136 prevents the engagement feature 174 from flexing radially outward in response to the energy source 110 urging the ram 108 distally. Rotation of the button 104 relative to the longitudinal axis L from the locked position to the unlocked position may align the button aperture 140 and the engagement feature 174. As discussed above, rotation of the button 104 relative to the longitudinal axis L may move the ridge 154 of the fin 152 from the locking detent 170a to the unlocking detent 170b. At least a portion of the fin 152 may engage the crown 168 when the button 104 is in the locked position and when the button 104 is in the unlocked position.

Referring to FIGS. 9-10, movement of the button 104 distally along the longitudinal axis L relative to the housing from the pre-firing position into the firing position may allow the ram 108 to move relative to the latch 106. Movement of the button 104 into the firing position may cause the button aperture 140 to overlap the engagement feature 174. The energy source 110 urging the ram 108 distally may force the engagement feature 174 to flex radially outward. Distal movement of the button 104 into the firing position may allow the engagement feature 174 to flex radially outward and extend at least partially through the button aperture 140. The ram 108 is free to move distally relative to the latch 106 when the engagement feature 174 is received in the button aperture 140.

The ram 108 may engage the plunger 128, thereby moving the plunger 128 relative to the medicament container 102 when the button is moved into the firing position. Movement of the plunger 128 relative to the medicament container 102 may eject the volume of the medicament from the medicament container 102 through the flange 130. The medicament may be expelled through a needle 132 when the needle 132 is coupled to the flange 130.

The button 104 may be prevented from moving proximally relative to the housing 100 after the button 104 has been moved from the pre-firing position to the firing position. The injector 10 may be single-use injector. The radial outward flex of the engagement feature 174 may prevent the button 104 from moving proximally along the longitudinal axis L when the button 104 is in the firing position.

A method of delivering a medicament to a subject using an injector 10 may be provided herein. The injector 10 may include a housing 100 having a proximal end and a distal end extending along a longitudinal axis L, a medicament container 102 containing a volume of a medicament, a button 104 coupled to the housing 100, the button 104 rotatable about the longitudinal axis L between a locked position and an unlocked position and movable along the longitudinal axis between a pre-firing position and a firing position, a latch 106 at least partially disposed within the button 104, a ram 108 movable relative to the latch 106, and an energy source 110 configured to urge the ram 108 distally along the longitudinal axis L.

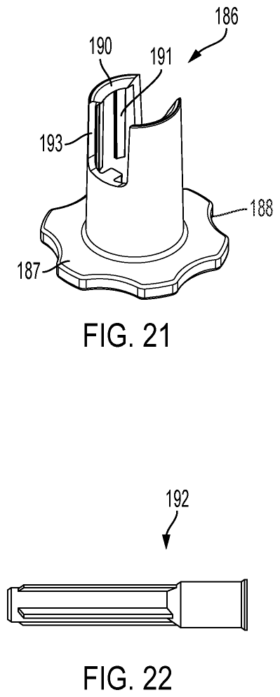

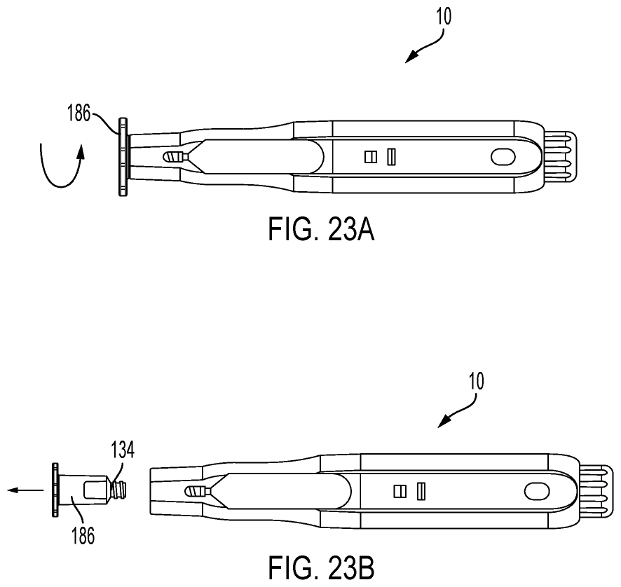

As shown in FIG. 23, the method may include decoupling a luer cap 134 from a flange 130 of the medicament container 102 with a luer cap remover 186. As discussed above, the luer cap 134 may be fully recessed within the housing 100. As shown in FIG. 21, the luer cap remover 186 may include a flange 187 extending radially outward from an end of the luer cap remover 186. The flange 187 may be a generally circular member with a diameter greater than a diameter of the luer cap remover 186 The flange 187 may prevent the luer cap remover 186 from being fully disposed within the housing 100. The flange 187 may include one or more user engagement features 188 defined on an outer end thereof. The user engagement feature 188 may provide a surface for a user to more easily grip and rotate the luer cap remover 186. The user engagement features 188 may be indents on the outer end of the flange 187. In some embodiments, the user engagement features extend from the outer end of the flange 187. The end of the luer cap remover 186 opposite the user engagement feature 188 may include a luer cap engager 190. The luer cap engager 190 may be shaped and sized to allow the luer cap 134 to be received therein. The luer cap engager 190 may include at least one rib 191 extending radially inward from an interior of the luer cap engager 190 to frictionally engage the luer cap 134 when the luer cap 134 is disposed therein. The luer cap engager 190 may include a slot 193 defined along a length thereof. The slot 193 may be a generally rectangular shape and may allow for the ends of the luer cap engager 190 to flex radially inward or outward when disposed in the housing 100 to accommodate the luer cap 134. The luer cap remover 186 may allow a user to rotate the luer cap 134 to remove the luer cap 134 from the flange 130.

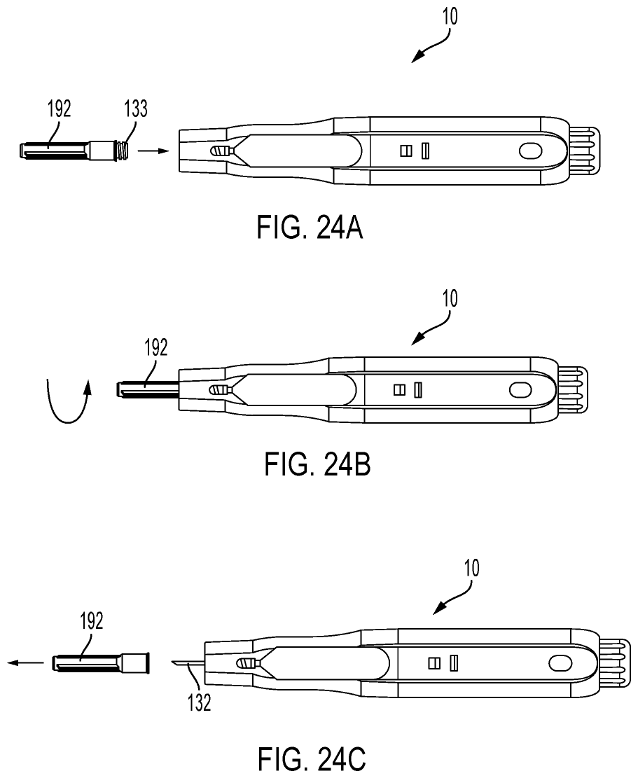

As shown in FIG. 24, the method may include coupling a needle 132 to the flange 130. The needle 132 may be in fluid communication with the medicament container 102 when the needle 132 is coupled thereto. The needle 132 may include a needle shield 192 coupled thereto. The needle shield 192 may provide sharps protection and prevent unintentional needle insertions. The needle shield 192 may include one or more ribs allowing a user to handle and rotate the needle 132 to couple the needle 132 to the flange 130. The needle shield 192 may be removed from the needle, as shown in FIG. 24 so expose the needle 132 for use.

The method may include rotating the button 104 to the unlocked position. As discussed above, rotating the button 104 in the unlocked position may align the button aperture 140 of the button 104 with the engagement feature 174 of the latch 106. Once moved into the unlocked position, the button 104 may be in the pre-firing position. The ram 108 may be prevented from moving relative to the latch 106 by the button 104 in the pre-firing position.

The method may include inserting at least a portion of the needle 132 into the subject. The needle 132 may be inserted into an insertion depth. Once the needle 132 has been inserted into the subject, the method may include moving the button 104 distally relative to the housing 100 into the firing position. In some embodiments, rotation of the button 104 to the unlocked position and movement of the button 104 distally along the longitudinal axis L relative to the housing 100 into the firing position may allow the ram 108 to move relative to the latch 106. Moving the button 104 distally into the firing position may allow the engagement feature 174 to flex radially outward and extend at least partially through the button aperture 140 by overlapping the button aperture 140 and the engagement feature 174.

The method may include ejecting the volume of the medicament through the needle 132 into the subject. The ram 108 may engage the plunger 128 disposed in the medicament container 102, thereby moving the plunger 128 relative to the medicament container 102 when the button 104 is moved into the firing position. In some embodiments, moving the plunger 128 relative to the medicament container 102 ejects the volume of the medicament from the medicament container 102 through the needle 132. The button 104 may be prevented from moving proximally relative to the housing 100 after the button 104 is moved into the firing position. The method may include removing the needle 132 from the subject after the medicament has been ejected therethrough. A user may dispose of the injector 10 after use.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

1. An injector comprising:
a housing having a proximal end and a distal end extending along a longitudinal axis;
a medicament container containing a volume of a medicament;
a button having a button body, a button aperture extending through the button body, and a fin extending distally from a proximal end of the button body coupled to the housing, the button rotatable about the longitudinal axis between a locked position and an unlocked position and movable along the longitudinal axis between a pre-firing position and a firing position;
a latch having a latch body, a crown defined on a proximal end of the latch body configured to engage the fin of the button, and an engagement feature extending from the latch body, the engagement feature movable between an engaged position and a disengaged position at least partially disposed within the button;
a ram movable relative to the latch; and
an energy source configured to urge the ram distally along the longitudinal axis,
wherein the ram is prevented from moving relative to the latch by the button in the pre-firing position,
wherein rotation of the button into the unlocked position aligns the button aperture with the engagement feature, and
wherein rotation of the button to the unlocked position and movement of the button distally along the longitudinal axis relative to the housing into the firing position allows the ram to move relative to the latch.

2. (canceled)

3. (canceled)

4. The injector of claim 1, wherein at least a portion of the fin engages the crown when the button is in the locked position and in the unlocked position.

5. The injector of claim 1, wherein the ram includes a ram body, and
a ram aperture extending through the ram body, the ram aperture configured to receive at least a portion of the engagement feature when the button is in the pre-firing position.

6. The injector of claim 1, wherein the engagement feature comprises:
a flexible arm extending along the longitudinal axis; and
a protrusion extending radially therefrom.

7. The injector of claim 6, wherein the protrusion extends radially inward from an end of the flexible arm.

8. (canceled)

9. The injector of claim 1, wherein distal movement of the button into the firing position allows the engagement feature to flex radially outward and extend at least partially through the button aperture.

10. The injector of claim 1, wherein the medicament container includes a luer lock extending from a distal end thereof, the injector further comprising:
a plunger disposed within the luer lock.

11. The injector of claim 10, wherein the ram engages the plunger thereby moving the plunger relative to the medicament container when the button is moved into the firing position.

12. The injector of claim 11, wherein movement of the plunger relative to the medicament container ejects the volume of the medicament from the medicament container through the luer lock.

13. The injector of claim 10 further comprising:
a needle coupled to the luer lock, the needle in fluid communication with the medicament container.

14. The injector of claim 10 further comprising:
a luer cap removably coupled to the luer lock in a storage configuration, wherein the luer cap is fully recessed within the housing.

15. The injector of claim 1, wherein the button is prevented from moving proximally relative to the housing after the button is moved from the pre-firing position to the firing position.

16. The injector of claim 1, wherein the volume of the medicament is at least 10 mL

17. The injector of claim 1, wherein the latch is fixed relative to the housing, and wherein the medicament container is fixed relative to the housing.

18.-26. (canceled)

27. An injector comprising:
a housing having a proximal end and a distal end extending along a longitudinal axis;
a medicament container having a luer lock extending from a distal end thereof and containing a volume of a medicament;
a button coupled to the housing, the button rotatable about the longitudinal axis between a locked position and an unlocked position and movable along the longitudinal axis between a pre-firing position and a firing position;
a latch at least partially disposed within the button;
a ram movable relative to the latch;
a plunger disposed within the medicament container; and
an energy source configured to urge the ram distally along the longitudinal axis,
wherein the ram is prevented from moving relative to the latch by the button in the pre-firing position, and
wherein rotation of the button to the unlocked position and movement of the button distally along the longitudinal axis relative to the housing into the firing position allows the ram to move relative to the latch.

28. The injector of claim 27, wherein the ram engages the plunger thereby moving the plunger relative to the medicament container when the button is moved into the firing position.

29. The injector of claim 28, wherein movement of the plunger relative to the medicament container ejects the volume of the medicament from the medicament container through the luer lock.

30. The injector of claim 27 further comprising:
a needle coupled to the luer lock, the needle in fluid communication with the medicament container.

31. The injector of claim 27 further comprising:
a luer cap removably coupled to the luer lock in a storage configuration,
wherein the luer cap is fully recessed within the housing.

32. An injector comprising:
a housing having a proximal end and a distal end extending along a longitudinal axis;
a medicament container containing a volume of a medicament, the medicament container fixed relative to the housing;
a button coupled to the housing, the button rotatable about the longitudinal axis between a locked position and an unlocked position and movable along the longitudinal axis between a pre-firing position and a firing position;
a latch at least partially disposed within the button, the latch fixed relative to the housing;
a ram movable relative to the latch; and
an energy source configured to urge the ram distally along the longitudinal axis,
wherein the ram is prevented from moving relative to the latch by the button in the pre-firing position, and
wherein rotation of the button to the unlocked position and movement of the button distally along the longitudinal axis relative to the housing into the firing position allows the ram to move relative to the latch.
